(19) United States
(12) Patent Application Publication
(10) Pub. No.: US 2015/0132398 A1
(43) Pub. Date: May 14, 2015

(54) VACCINE FORMULATION OF MANNOSE COATED PEPTIDE PARTICLES

(71) Applicants: CHARLES VINCENT TAYLOR HERST, Oakland, CA (US); REID M. RUBSAMEN, Alamo, CA (US)

(72) Inventors: CHARLES VINCENT TAYLOR HERST, Oakland, CA (US); REID M. RUBSAMEN, Alamo, CA (US)

(73) Assignee: FLOW PHARMA, INC., East Palo Alto, CA (US)

(21) Appl. No.: 14/601,849

(22) Filed: Jan. 21, 2015

Related U.S. Application Data
(63) Continuation of application No. 13/417,996, filed on Mar. 12, 2012.
(60) Provisional application No. 61/451,972, filed on Mar. 11, 2011.

(51) Int. Cl. A61K 9/16; A61K 39/00

(52) U.S. Cl. CPC A61K 9/167; A61K 39/00; A61K 9/1647; A61K 9/1623; A61K 2039/575; A61K 2039/64
USPC 424/499; 424/184.1

(57) ABSTRACT

A vaccine formulation as disclosed which is comprised of a pharmaceutically acceptable carrier in a plurality of particles with mannose on their surface. The particles are comprised of a biocompatible polymer which maybe a co-polymer such as PLGA combined with a peptide of a sequence which corresponds to a sequence on a surface of a pathogen. A plurality of different groups of particles are provided in the formulation wherein the particles within any single group include peptides of identical amino acid sequence. The particles are sized such that they are sufficiently large so as to prevent more than a single particle from being presented to a single immune system cell.

### FIELD OF THE INVENTION

This invention relates generally to the field of vaccines, and more particularly to vaccine formulations comprised of groups of particles, where the particles have mannose on an exposed outer surface and particles of a single group consist only of the same peptide species.

### BACKGROUND OF THE INVENTION

The term vaccine derives from Edward Jenner's 1796 use of the term cow pox (Latin variolæ vaccinæ, adapted from the Latin vaccīn-us, from vacca cow), which, when administered to humans, provided them protection against smallpox.

The 20th century saw the introduction of several successful vaccines, including those against diphtheria, measles, mumps, and rubella. Major achievements included the development of the polio vaccine in the 1950s and the eradication of smallpox during the 1960s and 1970s. Maurice Hilleman was the most prolific of the developers of the vaccines in the twentieth century. As vaccines became more common, many people began taking them for granted. However, vaccines remain elusive for many important diseases, including malaria and HIV. Vaccines may be dead or inactivated organisms or purified products derived from them.

There are several types of vaccines currently in use. These represent different strategies used to try to reduce risk of illness, while retaining the ability to induce a beneficial immune response. Considerable efforts have been made to develop an HIV vaccine.

The HIV virus has over 3,000 distinct epitopes on its surface. Each epitope can be targeted by the cellular immune response mediated by T-lymphocytes. These T-lymphocytes become sensitized to specific epitopes by exposure to antigens brought to the T-cells by antigen presenting cells (e.g macrophages).

HIV vaccines have been developed to direct cellular immunity mechanisms toward a blood borne HIV virus by sensitizing T-cells, via antigen presenting cells (APCs) exposed to the vaccine, to suites of epitopes on the surface of the virus.

Vectors used to introduce vaccines into the cellular immunity pathways have included adenovirus vectors. A problem with traditional vaccine approaches to treating patients already infected with HIV has been the fact that adenovirus vectors tend to activate CD4⁺ T-cells which in turn can potentially make pre-existing HIV infection more virulent. Another problem with HIV vaccine designs, in general, has been that the end result is to target large suites of epitopes on the surface of the virus, possibly targeting epitopes which could actually worsen various pathological aspects of the HIV infection.

Until recently, little was known about the specific effect of targeting specific epitopes on the surface of the HIV virus. Each time a vaccine vector is given to a person with HIV disease, a number of epitopes are targeted, and a number of humoral responses are measured. Associating a specific response with a specific epitope has been essentially impossible from an analysis of a single vaccine administration. Data from the administration of multiple vaccines to multiple sets of HIV infected subjects with corresponding humoral responses could, in theory, allow the effects of the individual epitopes to be de-convolved, essentially through a very computational intensive cross-correlation exercise.

Recent work in the field has put modern super-computer technology to bear on this problem resulting in a list of putatitive individual pathogen-relevant effects of individual epitopes on the surface of the HIV virus. Salient features of the analysis include:

1. Targeting some epitopes render the virus less pathogenic and targeting other epitopes make the virus less able to replicate.

2. Targeting other epitopes have the opposite effect making a virus more pathogenic and more able to replicate.

3. Regarding HIV, indications suggest approximately 30 unique epitopes should be targeted simultaneously to render the HIV virus substantially benign. Selecting the 30 preferred epitopes includes an expectation, based on vaccine clinical trial data analysis, that the virus will be “trapped” in an essentially non-pathogenic state, unable to mutate into another configuration.

A problem with traditional vaccine design is that there is no known way to design a single protein for presentation to an APC that will result in the targeting large numbers of epitopes on any pathogen, e.g. 30 epitopes on the HIV surface. It has been recognized however, that individual peptides of 9-11 amino acids contain enough structural specificity to create an APC event resulting in the targeting of a single unique epitope.

Placing protein antigens in PLGA microspheres of 100 nm-2 micron diameter can result in a prolonged, several day, presentation of the antigen to the immune system.

Results from antigen-containing PLGA microspheres made from a double-emulsion process utilizing organic solvents have been mixed, perhaps owing to the fact that the solvent systems and shear forces used in such microsphere fabrication processes can cause protein conformational changes perhaps interfering with the antigen-presenting event.

Peptides injected into the lymph system can be taken up by APC's producing an immune response. If a single APC takes up more than one antigen and simultaneously presents multiple antigens to T-cells, this may result in a cellular immune response wherein antibodies are produced with respect to only one antigen developed.

Methods for relieving the effects of immunodominance are described in published US patent application 20080260780, entitled “Materials And Methods Relating To Improved Vaccination Strategies”; US patent application 20090269362, entitled “Method for Controlling Immunodominance”; and US patent application 20100119535, entitled “Compositions and Methods for Immunodominant Antigens.”

### SUMMARY OF THE INVENTION

Inclusion of DC-SIGN-binding molecules, such as mannose or a molecule that includes mannose or an isomer thereof, into a solid matrix—such as a microparticle that contains one or more antigenic compounds for the purposes of stimulating an immune response for prophylactic (i.e., vaccine-related) purposes—to enhance the uptake of these microparticles by dendritic cells, macrophages and other DC-SIGN-expressing cells.

A vaccine composition is disclosed which is comprised of a plurality of groups of particles which may be substantially spherical mannose coated particles comprised of a polymer and a peptide which is an epitope. All of the particles in all of the groups have a particle size range such that only a single particle can be consumed by a mcrophage, e.g. the particle size may be in a range of 4 microns to 32 microns in diameter. The particles sizes may be from 6 microns to 16 microns, 8 microns to 12 microns in diameter. Although the size of the particles may vary over these ranges the particle sizes in the vaccine formulation may be substantially the same size ±20% or ±10%, ±2%, or ±1%.

Each particle comprises a biocompatible polymer with mannose on its surface and one or more peptides (of identical sequence) which may be at least 8 and not more than 20 amino acids, not more than 15, or not more than 11 amino acids in length. The peptides present in or on any one particle are identical to all other peptides on that particle, i.e. the peptides have the same amino acid sequence and consist of a single species. The particles may be provided as a group, i.e. a composition or formulation of single peptide species, or particles may be combined as a composition or formulation comprising particles with a plurality of peptide species. Compositions or groups of interest for manufacturing purposes, in kits, and the like may comprise single peptide species. Compositions and formulations for therapeutic purposes, e.g. for use in a method of vaccination, generally include at least two peptide species, and may include 3, 4, 5, 10, 20, 30, 40, 50 or more peptide species.

The composition containing the plurality of groups of particles may be a vaccine formulation which may have the particles in a dry form to which a liquid carrier can be added or the liquid carrier may be present and the liquid carrier may be a pharmaceutically acceptable injectable carrier.

The invention also includes a method of vaccinating a subject whereby a formulation of the invention is injected into an individual in order to elicit an immune response.

The invention also includes a method of making the particles and the population of groups of particles in order to form the formulation.

The vaccine formulations of the invention comprise polypeptides that correspond to an epitope, usually a T cell epitope, that, when brought into contact with a mammalian immune system will elicit an immune response to the corresponding epitope on a cell, virus, etc. Epitopes of interest include, without limitation, those present on pathogens, e.g. virus, bacteria, fungi, protozoan, etc.; and may further comprise epitopes associated with, for example, cancer cells. The polypeptide epitopes are encapsulated within and/or bound to the surface of specifically sized particles, where the particles may be formed of any suitable biocompatible material, e.g. biocompatible polymers such as poly(lactic-co-glycolic acid) (PLGA), polycaprolactone, polyglycolide, polylactic acid, poly-3-hydroxybutyrate, etc.

The invention provides optimally sized particles comprised of mannose, a biocompatible polymer, wherein the particles are loaded with a population of identical peptides, usually peptides that provide for a single epitope, and further wherein the particles are sized so that only a single particle for single population of peptides of identical amino acid sequence is presented to a single APC at a given point in time.

Another aspect of the invention is to provide a process whereby the particles as described above are formed by extrusion from a nozzle in a manner which creates particles and does not damage the peptides.

Another aspect of the invention is to provide such a process for producing particles which can be carried out without the use of solvents including organic solvents.

Another aspect of the invention is to obtained enhanced uptake of any type of particle intended to generate an immune response by including mannose or an isomer thereof on an exposed surface of the particle.

The invention includes various aspects including a formulation, a composition, a vaccine composition and use of the composition, formulation or vaccine composition in the manufacture of a medicament for use in a method of immunization.

The formulation comprises a pharmaceutically acceptable carrier and a plurality of particles comprised of a biocompatible polymer wherein each particle comprises a surface attached to a molecule comprised of mannose. The particles may be substantially spherical in shape and have a diameter in a range of about 4 microns to about 16 microns±20% (with no smaller or larger particles present).

The molecule comprised of mannose attached to the particles may consist of mannose, consist only of mannose and be mannose by itself positioned on a surface of the particles in a manner which allows binding of the mannose to a biological receptor. The biological receptor may be any molecular configuration on the surface of a cell or virus and the cell or virus may be a pathogen and as such be pathogenic bacteria and pathogenic viruses.

The composition of the invention comprises a plurality of groups of substantially spherical particles. The particles in each of the plurality of groups may have a diameter of about 4 microns to 32 microns (with no smaller or larger particles present) and be comprised of a biocompatible polymer and a peptide where each particle has a surface which is attached to mannose and the attachment may be covalent, ionic, or by physically entrapping the mannose molecule.

Within the composition the particles within the groups of the plurality of groups are identical to each other or substantially identical to each other in terms of the protein present in the particles. However, the different groups of particles are different from each other in terms of the protein associated with each different group. For purposes of the invention a plurality of groups may mean 2, 3, 4, 5, 10, 20, 30, etc. or more groups.

Within the formulation or the composition of the invention the biocompatible polymer may be a polymer which is selected from the group consisting of poly(lactic-co-glycolic acid) (PLGA), polycaprolactone, polyglycolide, polylactic acid, poly-3-hydroxybutyrate and the biological receptor is a surface receptor on a pathogen. Further, within the formulations and compositions of the invention the mannose may be a mannose isomer selected from the group consisting of α-D-Mannofuranose, β-D-Mannofuranose, α-D-Mannopyranose, and β-D-Mannopyranose.

The compositions and formulations of the invention may comprise an adjuvant which may be present by itself or added in combination with a pharmaceutically acceptable carrier.

In one aspect of the invention the adjuvant is particles of the composition in the absence of peptides or particles with peptides included within them. The peptides associated with the particles may be bound to the surface or embedded in the particles and particles within a given group may have the same size ±20% in diameter (with no smaller or larger particles in the formulation).

An aspect of the invention includes use of any composition or any formulation of the invention in the manufacture of a medicament for the use in a treatment such as a method of immunization.

The invention includes vaccine compositions which vaccine compositions comprise any of the compositions or formulations of the invention as described above.

In one aspect of the invention the vaccine composition comprises five or more groups of spherical particles having a diameter of 4 microns to 32 microns, (with no smaller or larger particles in the formulation) the particles being comprised of (a) a biocompatible polymer; (b) a plurality of peptides; and (c) a sugar monomer of the aldohexas series of carbohydrates and in particular the sugar monomer is mannose.

In another aspect of the invention mannose or molecules which hold a mannose monomer are attached to a surface of a construct in order to enhance the uptake of the construct by a human biological function.

These and other objects, advantages, and features of the invention will become apparent to those persons skilled in the art upon reading the details of the formulations and methods of treatment as more fully described below.

### DETAILED DESCRIPTION OF THE INVENTION

Before the present composition, vaccine, formulation and method of manufacture and use are described, it is to be understood that this invention is not limited to particular embodiment described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term ‘mannose’ is intended to cover all forms and isomers of mannose including optical isomers, D-mannose and L-mannose, isolated isomers and racemic mixtures.

A vaccine is a biological preparation that improves immunity to a particular disease. A vaccine typically contains an agent that resembles a disease-causing microorganism, and is often made from weakened or killed forms of the microbe or its toxins. The agent stimulates the body's immune system to recognize the agent as foreign, destroy it, and “recognize” it, so that the immune system can more easily recognize and destroy any of these microorganisms that it later encounters. Vaccines can be prophylactic (e.g. to prevent or ameliorate the effects of a future infection by any natural or “wild” pathogen), or therapeutic (e.g. vaccines against cancer are also being investigated).

The expression “enhanced immune response” or similar term means that the immune response is elevated, improved or enhanced to the benefit of the host relative to the prior immune response status, for example, this response is improved related to the native status before the administration of an immunogenic composition of the invention.

The terms “humoral immunity” and “humoral immune response” refer to the form of immunity in which antibody molecules are produced in response to antigenic stimulation.

The terms “cell-mediated immunity” and “cell-mediated immune response” are meant to refer to the immunological defense provided by lymphocytes, such as that defense provided by T cell lymphocytes when they come into close proximity to their victim cells. A cell-mediated immune response normally includes lymphocyte proliferation. When “lymphocyte proliferation” is measured, the ability of lymphocytes to proliferate in response to a specific antigen is measured. Lymphocyte proliferation is meant to refer to B cell, T-helper cell or cytotoxic T-lymphocyte (CTL) cell proliferation.

The term “immunogenic amount” refers to an amount of antigenic compound sufficient to stimulate an enhanced immune response, when administered with a subject immunogenic composition, as compared with the immune response elicited by the antigen in the absence of the microsphere formulation.

The terms “treatment”, “treating”, “treat” and the like are used herein to generally refer to obtaining a desired pharmacologic and/or physiologic effect such as an enhanced immune response. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a mammalian subject, more particularly a human, and includes: (a) preventing the disease or symptom from occurring in a subject which may be predisposed to the disease or symptom but has not yet been diagnosed as having it; (b) inhibiting the disease symptom, e.g., arresting its development; or relieving the disease symptom, i.e., causing regression of the disease or symptom (c) reduction of a level of a product produced by the infectious agent of a disease (e.g., a toxin, an antigen, and the like); and (d) reducing an undesired physiological response to the infectious agent of a disease (e.g., fever, tissue edema, and the like).

### INVENTION IN GENERAL

Most, if not all vaccines on the market today are soluble, i.e., are administered in liquid form, often with an adjuvant (a compound, such as BCG, to non-specifically stimulate the immune response). The antigen present in these soluble vaccines is intended to stimulate a specific immune response that may be boosted later on to be protective against a naturally occurring infection. Often, however, before such an antigen has the chance to interact with phagocytic cells of the immune system (i.e., dendritic cells, macrophages), the antigen dissipates, is destroyed in the body by enzymatic attack, and/or is cleared by the liver. What is needed is a method to stabilize the antigen, making it more resistant to attack, and making it more presentable to the macrophage.

One possible method of protecting such antigens is to encase them in a microparticle. These microparticles comprised of a biocompatible polymer can then be administered by a variety of routes (e.g., intravenous, intranasal) with the intention that macrophages will phagocytize these microparticles and process the antigens so they can be “presented” to other cells of the immune system. However, to be useful macrophages need to phagocytize these microparticles. If no macrophages are present in the area of the microparticle, the microparticle will eventually decay, releasing the antigen, resulting in a lack of the desired immune response. Thus, it would be useful to provide the antigen-containing microparticles with surface molecules that bind to the phagocytic cells so that the immune response is triggered.

Particles of the invention may be comprised of (1) a biocompatible polymer, (2) a peptide, and (3) mannose. The biocompatible polymer may be selected from the group consisting of poly(lactic-co-glycolic acid) (PLGA), polycaprolactone, polyglycolide, polylactic acid, poly-3-hydroxybutyrate. The peptide may have an amino acid sequence which matches that of a pathogen, which peptide may be three to fifty amino acids in length. The mannose may be physically embedded in the biocompatible polymer without a chemical connectors or chemically bound to the biocompatible polymer. At least some of the mannose molecules may be positioned on a surface of the particle such that the mannose is recognized by DC-SIGN and or DC-SGNR which may be on a macrophage.

DC-SIGN (Dendritic Cell-Specific Intercellular adhesion molecule 3-Grabbing Nonintegrin, Genbank accession number AF209479) and DC-SIGNR (DC-SIGN Related, Genbank accession number AF245219) are type II membrane proteins with close sequence homologies (77% identity in amino acids). DC-SIGN is expressed at high levels on both macrophages and dendritic cells; DC-SIGNR is expressed at high levels in liver and lymph nodes but not on dendritic cells; and both molecules are expressed on endometrium and placenta.

The DC-SIGN proteins are C-type (calcium-dependent) lectins. DC-SIGN on macrophages recognizes and binds to mannose-type carbohydrates, a class of pathogen-associated molecular patterns (PAMPs) commonly found on viruses, bacteria and fungi. For example, DC-SIGN and DC-SIGNR bind the HIV-1 surface envelope glycoprotein gp120 that contains a high number of mannose sugars, and this binding is inhibited by mannan. Both DC-SIGN and DC-SIGNR bind infectious HIV-1 particles and promote infection of susceptible T cells in trans. DC-SIGN and DC-SIGNR also avidly bind HCV envelope glycoproteins and thus serve as receptors for the virus. In addition, DC-SIGNR in particular is expressed at high levels in liver, the primary target organ for HCV infection. Since the ability of DC-SIGN and particularly DC-SIGNR to serve as receptors for HCV, the opportunity to treat or prevent HCV infection through therapies or vaccines that target these receptors. Microparticles containing both vaccine-related antigen and DC-SIGN-binding molecules, such as mannose, will thus bind to the DC-SIGN proteins on the surface of phagocytic cells, stimulating phagocytosis. This will allow the vaccine-related antigen present in the microparticles to be processed and presented by the cell to the other immune cells. Mannose is not immunogenic, nor is it an adjuvant (a non-specific method of stimulating all aspects of the immune response). Mannose specifically binds to DC-SIGN proteins on the phagocytic cells. Importantly, dendritic cells are the most potent of all the antigen-presenting cells, as dendritic cells can activate both memory and naïve T-cells.

Using the flow focusing technique, microparticles may be prepared using various biocompatible biodegradable polymers (e.g., poly-DL-lactide-co-glycolide) in combination with a vaccine-related antigen or antigens of choice (e.g., in the case of HIV, a peptide 10 amino acids in length) and a DC-SIGN-binding molecule, such as mannose. The microparticles so formed may be of 0.5-10 micron in size, with mannose and the vaccine-related antigen(s) on the surface of and internal to the microparticle itself. These particles may be rehydrated immediately before administration or may be administered as a dry powder, depending on the route of administration.

The formulation of the invention is designed such that a population of antigen-presenting cell (APC) can be presented with a plurality of peptide antigen species formulated in such a way that any one antigen presenting cell will present only a limited number of the peptide antigen species, i.e. less than 5, less than 3, usually presenting a single peptide species. It is believed that presentation of a plurality of epitopes by a single APC can result in immunodominance of a single epitope, which is undesirable in situations where responsiveness to the plurality of epitopes is optimal. For example, see Rodriguez, et al., “Immunodominance in Virus-Induced CD8+ T-Cell Responses Is Dramatically Modified by DNA Immunization and Is Regulated by Gamma Interferon” Journal of Virology, 76(9):4251-4259 (May 2002) and Yu et al., “Consistent Patterns in the Development and Immunodominance of Human Immunodeficiency Virus Type I (HIV-1)-Specific CD8+ T-Cell Responses following Acute HIV-1 Infection” Journal of Virology, 76(17):8690-9701 (September 2002), both incorporated herein by reference.

The invention accomplishes the desired result by placing a peptide antigen, as defined herein, on or in a particle of a defined size of a biocompatible polymer, usually in a form that is approved by the United States Food and Drug Administration for administration to humans. Vaccine formulation may be comprised of a pharmaceutically acceptable carrier. The carrier can come in a variety of forms depending on the mode of administration such as injection, nasal, inhalation, oral, etc. In addition to the carrier the formulation includes a plurality of particles which are comprised of a biocompatible polymer and a peptide. The peptide particles may be generally spherical in shape and have a defined diameter in a range of 4 microns to 16 microns. However, the diameter of the particles is designed such that an antigen presenting cells such as microphage can consume only a single particle. Each particle may contain a large number of peptides. However, the peptides within any given particle are identical. Thus, the particles consist only of peptides which have the same amino acid sequence. In some circumstances peptides may be allowed to include more than a single species of peptide, e.g. two, three, four or five peptides provided the peptides do not exhibit immunodominance with respect to each other.

Some examples of biocompatible polymers useful in the present invention include hydroxyaliphatic carboxylic acids, either homo- or copolymers, such as poly(lactic acid), poly(glycolic acid), Poly(dl-lactide/glycolide, poly(ethylene glycol); polysaccharides, e.g. lectins, glycosaminoglycans, e.g. chitosan; celluloses, acrylate polymers, and the like. The particle size is selected to (a) be capable of uptake and processing by an antigen presenting cell; and (b) be sufficiently large that an APC will generally take up not more than one particle.

Each set, or group, of particles comprises a single peptide species, i.e. peptides of identical sequences. The peptide antigen may be other than the sequence determined to be the immunodominant sequence. The particles may be provided as a group, i.e. a composition or formulation of single peptide species, or particles may be combined as a composition or formulation comprising particles with a plurality of peptide species, for example at least two peptide species, at least 3, at least 4, at least 5, at least 10, at least 20, and usually not more than 50, not more than 40, not more than 30 peptide species. In one embodiment, all of the particles are substantially spherical and have a diameter in a range of 4 microns to 16 microns. Each particle is comprised of a biocompatible polymer and with respect to peptides consists only of a single species of peptide, but may include many copies of that single species.

In some embodiments the peptide antigen (and optionally mannose) is embedded in the particle, for example by mixing peptides (and optionally mannose) and polymers prior formation of the particles. In other embodiments the antigens (and optionally mannose) are coupled to the particle surface.

Particles in a formulation may be heterogenous or homogenous in size, usually homogeneous, where the variability may be not more than 100% of the diameter, not more 50%, not more than 20%, not more than 10%, not more than 2%, etc. Particle sizes are may be about 4 μm in diameter, about 6 μm, about 8 μm about 10 μm, about 12 μm, about 14 μm, about 16, about 18 μm, about 20 μm, usually not more than about 32 μm, not more than about 25 μm diameter. The optimum size for a particular peptide or class of peptides may be determined empirically by various methods. For example, two different peptides may be detectably labeled with two different fluorophores, and used to prepare particles of the invention. A mixture of the particles is provided to antigen presenting cells, which are then viewed by eye, flow cytometry, etc. to determine if a single fluorophore or if multiple fluorophores are present in any single APC, where the size of particle that provides for exclusive uptake is chosen. Functional tests may also be performed, e.g. by providing particles with the cognate antigens for different T cell lines and determining if one or both lines are activated by an APC.

In order to determine the precise size which is desirable for the particles various types of labeling can be used. In addition to the fluorophores referred to above it is possible to use quantum dots. The purpose of carrying out the experiment is to determine a size at which the antigen presenting cells such as the macrophage can consume only a single particle. The size would be too large if the macrophage cannot consume the particle. The size would be too small if the macrophage can consume more than one particle.

The optimum size of particle to achieve the desired result may vary depending on the charge of the peptide that is being presented, for example a positively charged peptide may be more readily ingested by an APC than a neutral or negatively charged peptide. In some embodiments each peptide is individually optimized for a microsphere size that achieves exclusive uptake, and thus a formulation of a plurality of microsphere/peptide combinations may be heterogenous in size, although the size for a peptide species will be narrowly defined.

In some embodiments of the invention, a formulation is provided of PLGA microspheres of a defined size from 4 μm to 16 μm in diameter, where each microsphere comprises a single peptide antigen species, and where from 10 to 30 different peptide antigen species are present in the formulation. In some embodiments the peptide antigen is an antigen of the human immunodeficiency virus (HIV-1).

### Antigen Presenting Cells

The three major classes of antigen presenting cells are dendritic cells (DCs), macrophages, and B cells, but dendritic cells are considerably more potent on a cell-to-cell basis and are the only antigen presenting cells that activate naïve T cells. DC precursors migrate from bone marrow and circulate in the blood to specific sites in the body, where they mature. This trafficking is directed by expression of chemokine receptors and adhesion molecules. Upon exposure to antigen and activation signals, the DCs are activated, and leave tissues to migrate via the afferent lymphatics to the T cell rich paracortex of the draining lymph nodes. The activated DCs then secrete chemokines and cytokines involved in T cell homing and activation, and present processed antigen to T cells.

DCs mature by upregulating costimulatory molecules (CD40, CD80 and CD86), and migrate to T cell areas of organized lymphoid tissues where they activate naive T cells and induce effector immune responses. In the absence of such inflammatory or infectious signals, however, DCs present self-antigens in secondary lymphoid tissues for the induction and maintenance of self-tolerance. Dendritic cells include myeloid dendritic cells and plasmacytoid dendritic cells.

For purposes of the invention, e.g. determining the uptake of particles of vaccine formulations by APC, any one of the classes of APC may be used, including immature DC, monocytes, mature myeloid DC, mature pDC, etc. For example see Foged et al (2005) International Journal of Pharmaceutics 298(2): 315-322; Reece et al. (2001) Immunology and Cell Biology 79:255-263; Tel et al. (2010) J. Immunol. 184:4276-4283, each herein specifically incorporated by reference.

### Antigens

The term “antigen” as used herein includes meanings known in the art, and means a molecule or portion of a molecule, usually for the purposes of the present invention a polypeptide molecule, that can react with a recognition site on an antibody or T cell receptor. The term “antigen” also includes a molecule or a portion of a molecule that can, either by itself or in conjunction with an adjuvant or carrier, elicit an immune response (also called an “immunogen”).

The “specificity” of an antibody or T cell receptor refers to the ability of the variable region to bind with high affinity to an antigen. The portion of the antigen bound by the immune receptor is referred to as an epitope, and an epitope is that portion of the antigen which is sufficient for high affinity binding. An individual antigen typically contains multiple epitopes, although there are instances in which an antigen contains a single epitope. In some embodiments of the invention, a plurality of peptide fragments representing individual epitopes are derived from a protein antigen. Where the antigen is a protein, generally a linear epitope will be at least about 8 amino acids in length, and not more than about 15 to 22 amino acids in length. A T cell receptor recognizes a more complex structure than antibodies, and requires both a major histocompatibility antigen binding pocket and an antigenic peptide to be present. The binding affinity of T cell receptors is lower than that of antibodies, and will usually be at least about 10⁻⁴ M, more usually at least about 10⁻⁵ M.

Antigens of interest for the purposes of the invention include pathogens, e.g. virus, bacteria, protozoans, etc.; tumor antigens, and the like. Viral pathogens of interest include retroviral pathogens, e.g. HIV-1; HIV-2, HTLV, FIV, SIV, etc.; influenza, smallpox (vaccinia), measles, mumps, rubella, poliovirus, rotavirus, varicella (chickenpox), hepatitis A, B, C, D virus, bacterial antigens, tumor antigens, and the like. Microbes of interest, but not limited to the following, include: Citrobacter sp.; Enterobacter sp.; Escherichia sp., e.g. E. coli; Klebsiella sp.; Morganella sp.; Proteus sp.; Providencia sp.; Salmonella sp., e.g. S. typhi, S. typhimurium; Serratia sp.; Shigella sp.; Pseudomonas sp., e.g. P. aeruginosa; Yersinia sp., e.g. Y. pestis, Y. pseudotuberculosis, Y enterocolitica; Franciscella sp.; Pasturella sp.; Vibrio sp., e.g. V. cholerae, V. parahemolyticus; Campylobacter sp., e.g. C. jejuni; Haemophilus sp., e.g. H. influenzae, H. ducreyi; Bordetella sp., e.g. B. pertussis, B. bronchiseptica, B. parapertussis; Brucella sp., Neisseria sp., e.g. N. gonorrhoeae, N. meningitidis, etc. Other bacteria of interest include Legionella sp., e.g. L. pneumophila; Listeria sp., e.g. L. monocytogenes; Staphylococcus sp., e.g. S. aureus Mycoplasma sp., e.g. M. hominis, M. pneumoniae; Mycobacterium sp., e.g. M. tuberculosis, M. leprae; Treponema sp., e.g. T. pallidum; Borrelia sp., e.g. B. burgdorferi; Leptospirae sp.; Rickettsia sp., e.g. R. rickettsii, R. typhi; Chlamydia sp., e.g. C. trachomatis, C. pneumoniae, C. psittaci; Helicobacter sp., e.g. H. pylori, etc.

Antigenic peptides can include purified native peptides, synthetic peptides, recombinant proteins, crude protein extracts, attenuated or inactivated viruses, cells, micro-organisms, or fragments of such peptides. Immunomodulatory peptides can be native or synthesized chemically or enzymatically. Any method of chemical synthesis known in the art is suitable. Solution phase peptide synthesis can be used to construct peptides of moderate size or, for the chemical construction of peptides, solid phase synthesis can be employed. Atherton et al. (1981) Hoppe Seylers Z. Physiol. Chem. 362:833-839. Proteolytic enzymes can also be utilized to couple amino acids to produce peptides. Kullmann (1987) Enzymatic Peptide Synthesis, CRC Press, Inc. Alternatively, the peptide can be obtained by using the biochemical machinery of a cell, or by isolation from a biological source. Recombinant DNA techniques can be employed for the production of peptides. Hames et al. (1987) Transcription and Translation: A Practical Approach, IRL Press. Peptides can also be isolated using standard techniques such as affinity chromatography.

### Type of Vaccines

Some vaccines contain inactivated, but previously virulent, micro-organisms that have been destroyed with chemicals or heat. Examples are the influenza vaccine, cholera vaccine, bubonic plague vaccine, polio vaccine, hepatitis A vaccine, and rabies vaccine.

Some vaccines contain live, attenuated microorganisms. Many of these are live viruses that have been cultivated under conditions that disable their virulent properties, or which use closely-related but less dangerous organisms to produce a broad immune response, however some are bacterial in nature. They typically provoke more durable immunological responses and are the preferred type for healthy adults. Examples include the viral diseases yellow fever, measles, rubella, and mumps and the bacterial disease typhoid. The live Mycobacterium tuberculosis vaccine developed by Calmette and Guérin is not made of a contagious strain, but contains a virulently modified strain called “BCG” used to elicit immunogenicity to the vaccine.

Toxoid vaccines are made from inactivated toxic compounds that cause illness rather than the micro-organism. Examples of toxoid-based vaccines include tetanus and diphtheria. Toxoid vaccines are known for their efficacy. Not all toxoids are for micro-organisms; for example, Crotalus atrox toxoid is used to vaccinate dogs against rattlesnake bites.

Protein subunit—rather than introducing an inactivated or attenuated micro-organism to an immune system (which would constitute a “whole-agent” vaccine), a fragment of it can create an immune response. Examples include the subunit vaccine against Hepatitis B virus that is composed of only the surface proteins of the virus (previously extracted from the blood serum of chronically infected patients, but now produced by recombination of the viral genes into yeast), the virus-like particle (VLP) vaccine against human papillomavirus (HPV) that is composed of the viral major capsid protein, and the hemagglutinin and neuraminidase subunits of the influenza virus.

Conjugate—certain bacteria have polysaccharide outer coats that are poorly immunogenic. By linking these outer coats to proteins (e.g. toxins), the immune system can be led to recognize the polysaccharide as if it were a protein antigen. This approach is used in the Haemophilus influenzae type B vaccine.

A number of innovative vaccines are also in development and in use:
Dendritic cell vaccines combine dendritic cells with antigens in order to present the antigens to the body's white blood cells, thus stimulating an immune reaction. These vaccines have shown some positive preliminary results for treating brain tumors.
Recombinant Vector—by combining the physiology of one micro-organism and the DNA of the other, immunity can be created against diseases that have complex infection processes.
DNA vaccination—in recent years a new type of vaccine called DNA vaccination, created from an infectious agent's DNA, has been developed. It works by insertion (and expression, triggering immune system recognition) of viral or bacterial DNA into human or animal cells. Some cells of the immune system that recognize the proteins expressed will mount an attack against these proteins and cells expressing them. Because these cells live for a very long time, if the pathogen that normally expresses these proteins is encountered at a later time, they will be attacked instantly by the immune system. One advantage of DNA vaccines is that they are very easy to produce and store.
T-cell receptor peptide vaccines are under development for several diseases using models of Valley Fever, stomatitis, and atopic dermatitis. These peptides have been shown to modulate cytokine production and improve cell mediated immunity.
Targeting of identified bacterial proteins that are involved in complement inhibition would neutralize the key bacterial virulence mechanism.

While most vaccines are created using inactivated or attenuated compounds from micro-organisms, synthetic vaccines are composed mainly or wholly of synthetic peptides, carbohydrates or antigens.

Vaccines may be monovalent (also called univalent) or multivalent (also called polyvalent). A monovalent vaccine is designed to immunize against a single antigen or single microorganism. A multivalent or polyvalent vaccine is designed to immunize against two or more strains of the same microorganism, or against two or more microorganisms. In certain cases a monovalent vaccine may be preferable for rapidly developing a strong immune response.

In order to provide best protection, children are recommended to receive vaccinations as soon as their immune systems are sufficiently developed to respond to particular vaccines, with additional “booster” shots often required to achieve “full immunity”. This has led to the development of complex vaccination schedules. In the United States, the Advisory Committee on Immunization Practices, which recommends schedule additions for the Centers for Disease Control and Prevention, recommends routine vaccination of children against: hepatitis A, hepatitis B, polio, mumps, measles, rubella, diphtheria, pertussis, tetanus, HiB, chickenpox, rotavirus, influenza, meningococcal disease and pneumonia. The large number of vaccines and boosters recommended (up to 24 injections by age two) has led to problems with achieving full compliance. In order to combat declining compliance rates, various notification systems have been instituted and a number of combination injections are now marketed (e.g., Pneumococcal conjugate vaccine and MMRV vaccine), which provide protection against multiple diseases.

Besides recommendations for infant vaccinations and boosters, many specific vaccines are recommended at other ages or for repeated injections throughout life—most commonly for measles, tetanus, influenza, and pneumonia. Pregnant women are often screened for continued resistance to rubella. The human papillomavirus vaccine is currently recommended in the U.S. and UK for ages 11-25. Vaccine recommendations for the elderly concentrate on pneumonia and influenza, which are more deadly to that group. In 2006, a vaccine was introduced against shingles, a disease caused by the chickenpox virus, which usually affects the elderly.

### Producing Particles of the Formulation

Particles of the formulation could be produced in a number of different ways. However, it is desirable that the particles be produced with certain specific characteristics. For example, it is desirable that the particles in a formulation all have the same size ±20%, ±10%, ±5%, ±2%. It is also desirable that the process for producing the particles produce the particles without damaging the peptides of epitopes on the attenuated viruses in the particles. Such particles can be produced using a process referred to as “Flow Focusing” as disclosed within U.S. Pat. No. 6,116,516 issued Sep. 12, 2000 to Alfonso Ganan-Calvo.(incorporated here by reference).

Substantially any biocompatible polymer can be used in forming the particles and that polymer may be mixed with and/or bound to mannose. With respect to the peptide, the amino acid sequence in any given group of particles will be identical and will be chosen based on the particular pathogen of interest. Identical peptide sequences are produced and mixed with the desired polymer such as PLGA and the polymer with the peptides is extruded from a device (as disclosed within U.S. Pat. No. 6,116,516 which is incorporated herein by reference in its entirety) to produce particles.

In the present invention the epitope is generally only an amino acid sequence, but could be produced from a virus. Viruses are grown either on primary cells such as chicken eggs (e.g., for influenza), or on continuous cell lines such as cultured human cells (e.g., for hepatitis A). Bacteria are grown in bioreactors (e.g., Haemophilus influenzae type b). Alternatively, a recombinant protein derived from the viruses or bacteria can be generated in yeast, bacteria, or cell cultures. After the antigen is generated, it is isolated from the cells used to generate it. A virus may need to be inactivated, possibly with no further purification required. Recombinant proteins need many operations involving ultrafiltration and column chromatography. The vaccine may be formulated by adding adjuvant, stabilizers, and preservatives as needed. The adjuvant enhances the immune response of the antigen, stabilizers increase the storage life, and preservatives allow the use of multidose vials. Combination vaccines are harder to develop and produce, because of potential incompatibilities and interactions among the antigens and other ingredients involved.

Vaccine production techniques are evolving. Cultured mammalian cells are expected to become increasingly important, compared to conventional options such as chicken eggs, due to greater productivitity and few problems with contamination. Recombination technology that produces genetically detoxified vaccine is expected to grow in popularity for the production of bacterial vaccines that use toxoids. Combination vaccines are expected to reduce the quantities of antigens they contain, and thereby decrease undesirable interactions, by using pathogen-associated molecular patterns.

### Formulations

The compositions of the invention, especially useful to administering to an individual in need of immune stimulation (in the context of, for example, infectious disease, cancer, and allergy) generally comprise a plurality of microspheres of defined size comprising distinct peptide antigen species, as described herein in a sufficient amount to modulate an immune response.

The compositions of the invention optionally comprise a pharmaceutically acceptable excipient, and may be in various formulations. As is well known in the art, a pharmaceutically acceptable excipient is a relatively inert substance that facilitates administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington's Pharmaceutical Sciences 19th Ed. Mack Publishing (1995).

Generally, these compositions are formulated for administration by injection or inhalation, e.g., intraperitoneally, intravenously, subcutaneously, intramuscularly, etc. Accordingly, these compositions are preferably combined with pharmaceutically acceptable vehicles such as saline, Ringer's solution, dextrose solution, and the like. The particular dosage regimen, i.e., dose, timing and repetition, will depend on the particular individual and that individual's medical history.

In some embodiments, more than one antigen(s) may be present in a composition. Such compositions may contain at least two peptide species, at least 3, at least 4, at least 5, at least 10, at least 20, and usually not more than 50, not more than 40, not more than 30 peptide species. Such “cocktails”, as they are often denoted in the art, may be particularly useful in immunizing against pathogens present in different variants, e.g. HIV, rotavirus, influenza, etc. The cocktail formulation may be administered on time only or administered two, three or more times to obtain that desired immune response.

Generally, the efficacy of administering any of these compositions is adjusted by measuring any change in the immune response as described herein, or other clinical parameters.

In some embodiments, the invention provides compositions comprising polypeptides as described herein and an adjuvant whereby the polypeptide(s)/adjuvant are co-administered. The immunogenic composition may contain an amount of an adjuvant sufficient to potentiate the immune response to the immunogen. Adjuvants are known in the art and include, but are not limited to, oil-in-water emulsions, water-in oil emulsions, alum (aluminum salts), liposomes and microparticles including but not limited to, polystyrene, starch, polyphosphazene and polylactide/polyglycosides. Other suitable adjuvants also include, but are not limited to, MF59, DETOX™ (Ribi), squalene mixtures (SAF-1), muramyl peptide, saponin derivatives, mycobacterium cell wall preparations, monophosphoryl lipid A, mycolic acid derivatives, nonionic block copolymer surfactants, Quil A, cholera toxin B subunit, polyphosphazene and derivatives, and immunostimulating complexes (ISCOMs) such as those described by Takahashi et al. (1990) Nature 344:873-875, as well as, lipid-based adjuvants and others described herein. For veterinary use and for production of antibodies in animals, mitogenic components of Freund's adjuvant (both complete and incomplete) can be used.

In some embodiments, the plurality of microspheres or particles of defined size comprising distinct peptide antigen species described herein can be administered in conjunction with one or more immunomodulatory facilitators. Thus, the invention provides compositions comprising plurality of microspheres of defined size comprising distinct peptide antigen species and an immunomodulatory facilitator. As used herein, the term “immunomodulatory facilitator” refers to molecules which support and/or enhance immunomodulatory activity. Immunomodulatory facilitators include, but are not limited to, co-stimulatory molecules (such as cytokines, chemokines, targeting protein ligand, trans-activating factors, peptides, and peptides comprising a modified amino acid) and adjuvants (such as alum, lipid emulsions, and polylactide/polyglycolide microparticles).

The following excipients are commonly present in vaccine preparations:
Aluminum salts or gels are added as adjuvants. Adjuvants are added to promote an earlier, more potent response, and more persistent immune response to the vaccine; they allow for a lower vaccine dosage.
Antibiotics are added to some vaccines to prevent the growth of bacteria during production and storage of the vaccine.
Egg protein is present in influenza and yellow fever vaccines as they are prepared using chicken eggs. Other proteins may be present.
Formaldehyde is used to inactivate bacterial products for toxoid vaccines. Formaldehyde is also used to kill unwanted viruses and bacteria that might contaminate the vaccine during production.
Monosodium glutamate (MSG) and 2-phenoxyethanol are used as stabilizers in a few vaccines to help the vaccine remain unchanged when the vaccine is exposed to heat, light, acidity, or humidity.
Thimerosal is a mercury-containing preservative that is added to vials of vaccine that contain more than one dose to prevent contamination and growth of potentially harmful bacteria.

Many vaccines need preservatives to prevent serious adverse effects such as the Staphylococcus infection that, in one 1928 incident, killed 12 of 21 children inoculated with a diphtheria vaccine that lacked a preservative. Several preservatives are available, including thiomersal, phenoxyethanol, and formaldehyde. Thiomersal is more effective against bacteria, has better shelf life, and improves vaccine stability, potency, and safety, but in the U.S., the European Union, and a few other affluent countries, it is no longer used as a preservative in childhood vaccines, as a precautionary measure due to its mercury content. Although controversial claims have been made that thiomersal contributes to autism, no convincing scientific evidence supports these claims.

### Administration and Assessment of the Immune Response

A formulation of the invention may be comprised of a carrier and particles as described here. The particle may be of a single group where all the peptides in all the particle have the same peptide sequence. In this embodiment the formulation is administered and thereafter a formulation with a different peptide is administered. The number of different peptides and administrations can vary depending on the pathogen for which immunity is being sought. Thus, the number of administrations and different peptides can be one, two, three . . . thirty or more.

Alternatively a cocktail of different particles groups are administered. In accordance with this method all the peptides in any given particle are the same, but different from the peptide of another group. All the peptides in any single particle will be identical in an attempt to avoid issues of immunodominance (see J of Virology, May 2002, p. 4251-4259, Rodriguez at al.) incorporated here by reference.

The plurality of microspheres or particles of the invention of defined size comprising distinct peptide antigen species composition can be administered in combination with other pharmaceutical and/or immunogenic and/or immunostimulatory agents and can be combined with a physiologically acceptable carrier thereof.

As with all immunogenic compositions, the immunologically effective amounts and method of administration of the particular formulation can vary based on the individual, what condition is to be treated and other factors evident to one skilled in the art. Factors to be considered include the antigenicity, route of administration and the number of immunizing doses to be administered. Such factors are known in the art and it is well within the skill of immunologists to make such determinations without undue experimentation. A suitable dosage range is one that provides the desired modulation of immune response to the antigen. Generally, a dosage range may be, for example, from about any of the following, referencing the amount of peptide in a dose exclusive of carrier: 0.01 to 100 μg, 0.01 to 50 μg, 0.01 to 25 μg, 0.01 to 10 μg, 1 to 500 μg, 100 to 400 μg, 200 to 300 μg, 1 to 100 μg, 100 to 200 μg, 300 to 400 μg, 400 to 500 μg. Alternatively, the doses can be about any of the following: 0.1 μg, 0.25 μg, 0.5 μg, 1.0 μg, 2.0 μg, 5.0 μg, 10 μg, 25 μg, 50 μg, 75 μg, 100 μg. Accordingly, dose ranges can be those with a lower limit about any of the following: 0.1 μg, 0.25 μg, 0.5 μg and 1.0 μg; and with an upper limit of about any of the following: 250 μg, 500 μg and 1000 μg. The absolute amount given to each patient depends on pharmacological properties such as bioavailability, clearance rate and route of administration.

The effective amount and method of administration of the particular formulation can vary based on the individual patient and the stage of the disease and other factors evident to one skilled in the art. The route(s) of administration useful in a particular application are apparent to one of skill in the art. Routes of administration include but are not limited to topical, dermal, transdermal, transmucosal, nasal, oral, epidermal, parenteral, gastrointestinal, and naso-pharyngeal and pulmonary, including transbronchial and transalveolar. The absolute amount given to each patient depends on pharmacological properties such as bioavailability, clearance rate and route of administration.

Parenteral routes of administration include but are not limited to electrical (iontophoresis) or direct injection such as direct injection into a central venous line, intravenous, intramuscular, intraperitoneal, intradermal, or subcutaneous injection. Compositions suitable for parenteral administration include, but are not limited, to pharmaceutically acceptable sterile isotonic solutions. Such solutions include, but are not limited to, saline and phosphate buffered saline for injection of the compositions.

Naso-pharyngeal and pulmonary routes of administration include, but are not limited to, inhalation, transbronchial and transalveolar routes. The invention includes compositions suitable for administration by inhalation including, but not limited to, various types of aerosols for inhalation, as well as powder forms for delivery systems. Devices suitable for administration by inhalation of include, but are not limited to, atomizers and vaporizers. Atomizers and vaporizers filled with the powders are among a variety of devices suitable for use in inhalation delivery of powders.

The methods of producing suitable devices for injection, topical application, atomizers and vaporizers are known in the art and will not be described in detail.

The above-mentioned compositions and methods of administration are meant to describe but not limit the methods of administering the compositions of the invention. The methods of producing the various compositions and devices are within the ability of one skilled in the art and are not described in detail here.

Analysis (both qualitative and quantitative) of the immune response to the subject compositions can be by any method known in the art, including, but not limited to, measuring antigen-specific antibody production (including measuring specific antibody subclasses), activation of specific populations of lymphocytes such as CD4+ T cells or NK cells, production of cytokines such as IFNγ, IL-2, IL-4, IL-5, IL-10 or IL-12 and/or release of histamine. Methods for measuring specific antibody responses include enzyme-linked immunosorbent assay (ELISA) and are well known in the art. Measurement of numbers of specific types of lymphocytes such as CD4+ T cells can be achieved, for example, with fluorescence-activated cell sorting (FACS). Serum concentrations of cytokines can be measured, for example, by ELISA. These and other assays to evaluate the immune response to an immunogen are well known in the art. See, for example, Selected Methods in Cellular Immunology (1980) Mishell and Shiigi, eds., W.H. Freeman and Co.

In some instances, a Th1 or Th2-type response is stimulated, i.e., elicited and/or enhanced. With reference to the invention, stimulating a Th1 or Th2-type immune response can be determined in vitro or ex vivo by measuring cytokine production from cells treated with a composition of the invention as compared to those treated a conventional vaccine. Methods to determine the cytokine production of cells include those methods described herein and any known in the art. The type of cytokines produced in response to treatment indicates a Th1-type or a Th2-type biased immune response by the cells. As used herein, the term “Th1-type biased” cytokine production refers to the measurable increased production of cytokines associated with a Th1-type immune response in the presence of a stimulator as compared to production of such cytokines in the absence of stimulation. Examples of such Th1-type biased cytokines include, but are not limited to, IL-2, IL-12, and IFN-γ. In contrast, “Th2-type biased cytokines” refers to those associated with a Th2-type immune response, and include, but are not limited to, IL-4, IL-5, and IL-13. Cells useful for the determination of activity include cells of the immune system, primary cells isolated from a host and/or cell lines, usually APCs and lymphocytes.

There are several new delivery systems in development to make vaccine delivery more efficient. Methods include liposomes and ISCOM (immune stimulating complex).

Delivery Systems

Vaccine delivery technologies have resulted in oral vaccines. A polio vaccine was developed and tested by volunteer vaccinations with no formal training; the results were positive in that the ease of the vaccines increased dramatically. With an oral vaccine, there is no risk of blood contamination. Oral vaccines are likely to be solid which have proven to be more stable and less likely to freeze; this stability reduces the need for a “cold chain”: the resources required to keep vaccines within a restricted temperature range from the manufacturing stage to the point of administration, which, in turn, will decrease costs of vaccines. Finally, a microneedle approach, which is still in stages of development, seems to be the vaccine of the future, the microneedle, which is “pointed projections fabricated into arrays that can create vaccine delivery pathways through the skin”.

### METHODS OF THE INVENTION

The invention also includes methods of modulating an immune response comprising administering an immunogenic formulation as described herein to an individual in an amount sufficient to modulate the immune response. Generally, the individual is in need of, or will be in need of, such modulation, due, for example, for a disease condition or being at risk of developing a disease condition. Examples of disease conditions include, but are not limited to, allergy, cancer, infectious diseases (such as viral or bacterial infection).

It is to be understood that this invention is not limited to the particular methodology, protocols, peptides, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the reagents, cells, constructs, and methodologies that are described in the publications, and which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A formulation, comprising:
a pharmaceutically acceptable carrier;
a plurality of particles comprised of a biocompatible polymer, a single species of peptides, and mannose molecules selected from the group consisting of D-mannose and L-mannose wherein the peptides and mannose molecules are embedded in the biocompatible polymer and mannose molecules are positioned to allow binding of mannose to a biological receptor.

2. The formulation of claim 1, wherein the particles are substantially spherical.

3. (canceled)

4. The formulation of claim 2, wherein each particle has a diameter in a range of from 4 microns to 16 microns.

5. The formulation of claim 4, wherein the biocompatible polymer is selected from the group consisting of poly(lactic-co-glycolic acid) (PLGA), polycaprolactone, polyglycolide, polylactic acid, poly-3-hydroxybutyrate and the biological receptor is a surface receptor on a pathogen.

6. The formulation of claim 5, wherein the formulation comprises 100 or more particles and the pathogen is a virus.

7. (canceled)

8. A composition, comprising:
a plurality of groups of substantially spherical particles, which particles in each of the plurality of groups of particles are 4 microns to 32 microns in diameter and are comprised of a biocompatible polymer, a single species of peptides, and mannose molecules selected from the group consisting of D-mannose and L-mannose, wherein the peptides and mannose molecules are embedded in the biocompatible polymer and are positioned to allow binding of mannose to a biological receptor.

9. The composition of claim 8, wherein particles in a first group of the plurality of groups of particles comprise a peptide of an identical amino acid sequence to a peptide in all other particles in the first group of particles, and
wherein particles in a second group of the plurality of groups of particles comprise a peptide of an identical amino acid sequence to a peptide in all other particles in the second group of particles which amino acid sequence is different from the amino acid sequence of the peptide in the first group of particles.

10. The composition of claim 8, wherein the mannose is comprised of a mannose isomer selected from the groups consisting of α-D-Mannofuranose, β-D-Mannofuranose, α-D-Mannopyranose, and β-D-Mannopyranose.

11. The composition of claim 8, further comprising:
an adjuvant and a pharmaceutically acceptable carrier;
wherein each group of particles comprises 100 or more particles and the composition comprises five or more groups of particles wherein the particles of each group consists of an identical peptide which peptide is different from a peptide in any other group of particles.

12. The composition of claim 11, wherein the adjuvant is in particles of the composition and the peptides are dispersed throughout the particles.

13. The composition of claim 8, wherein the peptides are bound to the surface of the particles.

14. The composition of claim 8, wherein all the particles of the composition have the same diameter plus or minus 20%.

15.-18. (canceled)

19. An adjuvant formulation, comprising:
a pharmaceutically acceptable carrier;
a plurality of substantially spherical particles comprised of a biocompatible polymer, wherein each particle comprises mannose molecules selected from the group consisting of D-mannose and L-mannose, wherein the mannose molecules are embedded in the biocompatible polymer and are positioned to allow binding of mannose to a biolotical receptor positioned to allow binding of the mannose to a biological receptor;
wherein each particle has a diameter in a range of from 4 microns to 16 microns;
wherein the biocompatible polymer is selected from the group consisting of poly(lactic-co-glycolic acid) (PLGA), polycaprolactone, polyglycolide, polylactic acid, poly-3-hydroxybutyrate and the biological receptor is a surface receptor on a pathogen; and
wherein the formulation comprises 100 or more particles and the pathogen is a virus.

20. A vaccine particle, comprising:
a biocompatible polymer forming the particle structure;
an antigenic component embedded in the particle; and
a single species of peptides, and mannose molecules selected from the group consisting of D-mannose and L-mannose, wherein the peptides and mannose molecules are embedded in the biocompatible polymer and are positioned to allow binding of mannose to a biolotical receptor to allow binding of a pathogen to the mannose.

21. The vaccine particle of claim 20, wherein the particle is further comprised of an adjuvant.
